(19) United States
(12) Patent Application Publication
(10) Pub. No.: US 2022/0288112 A1
(43) Pub. Date: Sep. 15, 2022

(54) COMPOSITION FOR TREATMENT OF BURN WITH SCAR REMOVAL

(71) Applicant: Huxley JOSE, Kottayam (IN)

(72) Inventor: Huxley JOSE, Kottayam (IN)

(21) Appl. No.: 17/636,105

(22) PCT Filed: Aug. 7, 2020
(86) PCT No.: PCT/IN2020/050699

§ 371 Date: Feb. 17, 2022

(30) Foreign Application Priority Data
Sep. 6, 2019 (IN) 201911035978

(51) Int. Cl. A61K 33/241; A61K 47/44

(52) U.S. Cl. CPC A61K 33/241; A61K 47/44

(57) ABSTRACT

The present invention relates to composition and method for treatment of burn with scar removal. In this, powder of Shanku Padikaram is mixed with coconut oil and heated for 8 to 10 minutes. The mixture is prepared in a Bronze cauldron which is made up of bell metal. After cooling of mixture completely, it is applied over affected skin.

### FIELD OF THE INVENTION

The present invention relates to composition and method for the treatment of burn with scar removal. The composition is made up of Shanku Padikaram (Murdar Sang herbal stone) and coconut oil and prepared in a bronze cauldron made up of bell metal.

### BACKGROUND OF THE INVENTION

Burns have always been difficult to treat. They are caused by heat, cold, electricity, chemicals, friction or radiation. Burns that affect only the superficial skin layers are known as superficial or first-degree burns. First-degree burns appear red without blisters and pain in it lasts around three days. When the injury extends into underlying skin layer, it is a partial-thickness or second-degree burn. Blisters are frequently present in second-degree burns, and they are often very painful. Healing can require up to eight weeks and scarring may occur. In a full-thickness or third-degree burn, the injury extends to all layers of the skin. There is no pain and the burnt area is stiff in the third-degree burns. Healing does not occur on its own in third-degree burn. A fourth-degree burn involves injury to deeper tissues, such as muscle, tendons, or bone. The burn turns black and frequently leads to loss of the burned part in fourth-degree burns. The world's medical research for burn treatment is generally considered to be the beginning of the 1930s. The traditional medicine treatment of burns has 5,000 years of history.

Current treatments for burns usually take long time to heal burns, and to regenerate tissue and recover slowly, and the current medicines for the treatment of burns formula create side effects like bedsores, diabetic symptoms similar therapeutic effect gangrene ulcers, chronic ulcers etc.

FIG. 1 is a diagram showing different type of burns according to one or more embodiments of the present invention. According to one embodiment of the present invention, there are three categories of burns. The burns that affect only the superficial skin layers are known as superficial or first-degree burns. They appear red without blisters and pain typically lasts around three days. When the injury extends into some of the underlying skin layer, it is a partial-thickness or second-degree burn. Blisters are frequently present, and they are often very painful. Healing can require up to eight weeks and scarring may occur. In a full-thickness or third-degree burn, the injury extends to all layers of the skin. Often there is no pain and the burnt area is stiff. Healing typically does not occur on its own. A fourth-degree burn additionally involves injury to deeper tissues, such as muscle, tendons, or bone. The burn is often black and frequently leads to loss of the burned part. Many times, the skin also peels of leaving a white patch colour. The body produces a protein called collagen to repair the damaged skin, which leads to a scar. Scars caused by second and third-degree burns are categorized according to their appearance and how they affect the skin:
Hypertrophic scars may appear red or purple and are raised above the skin level. They may feel warm and become itchy.
Contracture scars make the skin, muscles, and tendons tighter and restrict normal skin movement.
Keloid scars form a shiny, hairless bump.

In one embodiment, block diagrams represent categories of burn. In first degree burn, only outermost layer of the skin (100) called epidermis 100a is affected. The epidermis is the outermost of the three layers that make up the skin, the inner layers being the dermis and hypodermis. The epidermis layer provides a barrier to infection from environmental pathogens and regulates the amount of water released from the body into the atmosphere through trans epidermal water loss. The epidermis is composed of multiple layers of flattened cells that overlie a base layer composed of columnar cells arranged perpendicularly. In second degree burn, infection extends into superficial papillary dermis layer 100b of the skin. The dermis or corium 100b is a layer of skin between the epidermis and subcutaneous tissues 100c, that primarily consists of dense irregular connective tissue and cushions the body from stress and strain. It is divided into two layers, the superficial area adjacent to the epidermis called the papillary region and a deep thicker area known as the reticular dermis. The dermis 100b is tightly connected to the epidermis 100a through a basement membrane. In third degree burn, infection extends through entire dermis.

US patent application U.S. Ser. No. 05/861,304 discloses a skin treatment composition or formulation comprising para-aminobenzoic acid, calcium-d-pantothenate, and a tocopherol compound, and desirably a gelling agent, is disclosed, along with methods of applying such compositions for use as a sun screening agent, or for the treatment of skin ailments, particularly burns. To such compositions, a waxy extract of Kava may be included as a local anaesthetic, along with a suitable carrier, such as a mixture of light unsaturated oils with stimulant agents.

Indian Patent Application IN1577/CHE/2007 discloses a composition for treatment of burns comprising coconut oil, sulphur, calcium and water by heating one Litre of coconut oil in low flame up to 20 to 30 degree centigrade in an aluminium container and adding 75 grams of Sulphur while continuing to heat in low flame until sulphur precipitates and losses Sulphur Di Oxide and extracting upper layer of the solution free of sulphur waste by transferring it into another vessel and the process of extracting calcium precipitate by mixing 20 Grams of lime into the ordinary water free of impurities and stirring until the calcium dissolves and extracting the upper layer of calcium water solution and mixing the precipitated coconut oil-sulphur solution and calcium precipitate water ready for application on skin surface.

The prior arts do not talk about healing of burn up to third degree and protecting the skin from discolouration. Therefore, there is a need in the art for a method and apparatus for providing methods and compositions for the treatment of burn that can cure burn up to third degree burn in a short span of time and can also protect skin from discolouration.

### SUMMARY OF THE INVENTION

Embodiments of the present invention composition and method for the treatment of burn with scar removal. In one embodiment, a composition for the treatment of burn comprising Shanku Padikaram and coconut oil is disclosed. In another embodiment, a method for the treatment of burn with removal of scar on skin is disclosed. The present invention addresses the shortcomings and problems mentioned in the prior arts and provides better method for cure of burn.

In one embodiment of the claimed invention, specific amount of Shanku Padikaram herb is crushed to a powder and is then mixed with some amount of coconut oil. Then mixture of Shanku Padikaram and coconut oil is boiled in a cauldron for a couple of minutes. The time period for boiling of mixture may extend from 8 to 10 minutes and the mixture after boiling is left to cool down completely. Then the cooled mixture is applied over affected area for 2 to 3 months depending upon the severity of the burn. The body produces a protein called collagen to repair the damaged skin, which leads to a scar. The claimed invention provides the compositions and methods which assists in the production of collagen, thereby reducing and or removing the scar from the skin entirely.

Moreover, the invention provides the medicine which remove hypertrophic scars as well as contracture scars entirely. It also removes keloid scars than 90% if applied for 2 to 3 months depending on the severity of the burn.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 2 is a diagram showing structure of container called cauldron used for the preparation of composition for treatment of burns according to one or more embodiments of the present invention;

FIG. 3 is a diagram showing the chemical composition of chemical compound called Shanku Padikaram used for the preparation of composition according to one or more embodiments of the present invention;

FIG. 4 is a flow diagram showing the method for the treatment of burn with scar removal according to one or more embodiments of the present invention.

FIG. 5 is an image showing small red rock like material called Shanku Padikaram.

### DETAILED DESCRIPTION OF EMBODIMENT

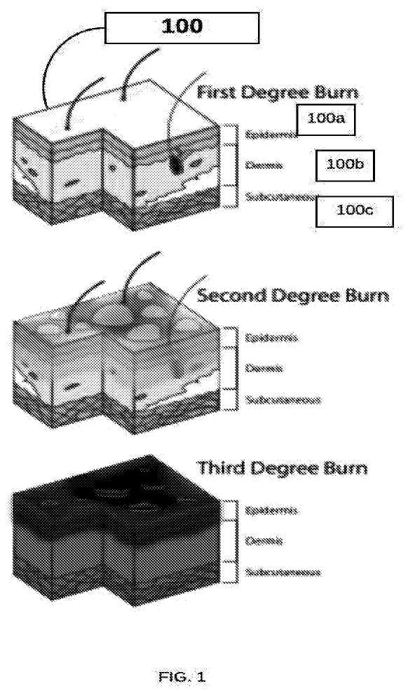

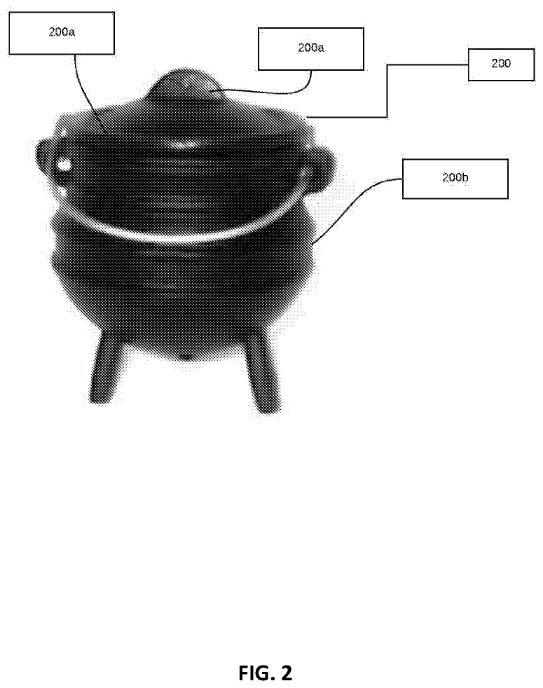

FIG. 2 is a diagram showing structure of a cauldron 200 used for the preparation of composition for treatment of burns according to one or more embodiments of the present invention. The cauldron used for the preparation of composition used in the claimed invention is made up of bell metal. The cauldron 200 has top broad oval shaped open mouth (200a), broad spherical bottom (200b) in which the composition is prepared, and a lid (200c) to cover the mouth of the cauldron. The pot is kept on fire and the mixture is allowed to boil and mix slowly for 8-10 minutes. Then the mixture is allowed to cool down completely. After cooling of mixture, the lid is kept on the mouth of the cauldron 200. The mixture is applied on the affected skin area for continuous 2 to 3 months depending on the severity of the burn.

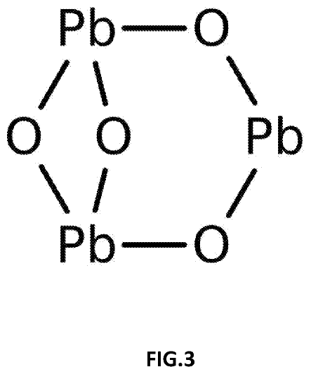

FIG. 3 is a diagram showing the chemical composition of herb called Shanku Padikaram used for the preparation of composition according to one or more embodiments of the present invention. Shanku Padikaram is a rock like material used for skin health and beauty purposes. It comes in solid rock form and powder form. Its scientific formula is Triplumbic Tetroxide. It is also known as Shanku Padiharam, red lead, Litharge, Murdar Singh, Murdar Sang, Murda Shank etc

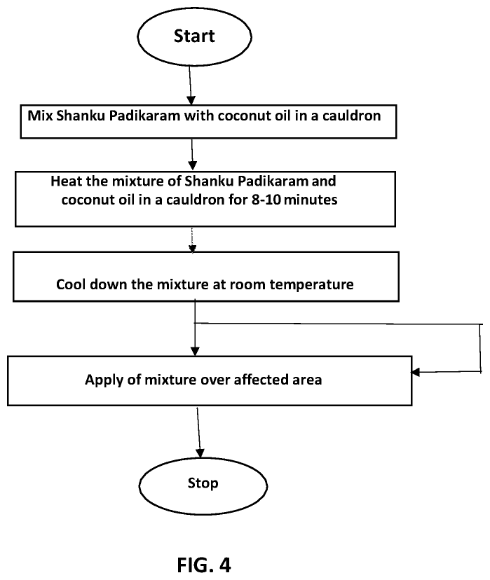

FIG. 4 is a flow diagram showing the method for the treatment of burn with scar removal according to one or more embodiments of the present invention. The method 400 starts at step 402 at which 12 grams of Shanku Padikaram is taken in a cauldron 200 and is crushed to powder. At step 404, powder of Shanku Padikaram is mixed with 360 ml of coconut oil. At step 406, mixture is boiled slowly for 8-10 minutes and is allowed to cool down completely. After cooling of mixture, the same can be applied over affected area. The step of application of mixture over affected area is repeated for 2 to 3 months depending upon the severity of the burn. The method 400 ends at step 408.

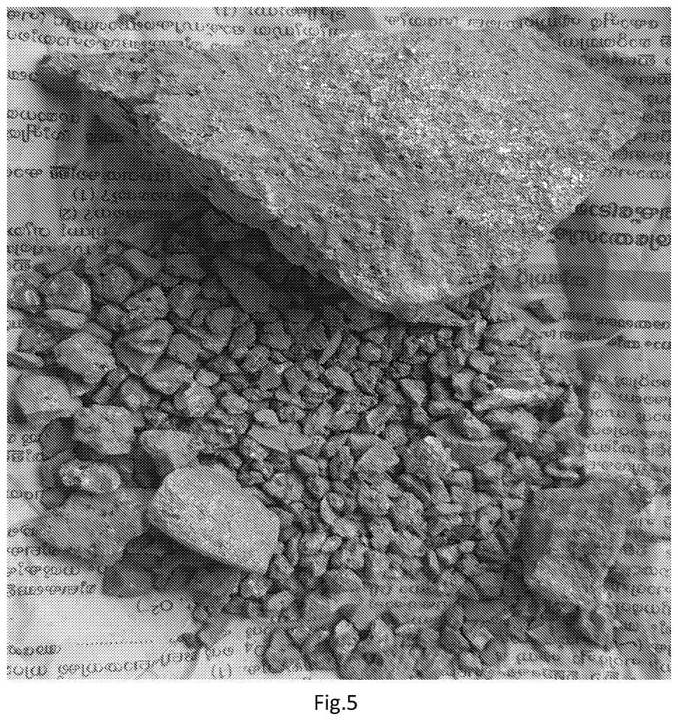

FIG. 5 is an image of compound used in the aforementioned composition. The compound used is Shanku Padikaram. It is used in Ayurvedic and Herbal medicines. The chemical formula of said compound is Triplumbic Tetroxide. It comes in the form red small stones. For easy reference, image of compound is shown. [0021] While the foregoing is directed to embodiments of the present invention, other and further embodiments of the invention may be devised without departing from the basic scope thereof, and the scope thereof is determined by the claims that follow.

1. A composition for treatment of burn with scar removal comprising Shanku Padikaram herbal powder and coconut oil, wherein the composition is capable of curing burn of third degree.

2. The composition as claimed in claim 1, wherein the amount of Shanku Padikaram herb in the composition between about 12-20 grams.

3. The composition as claimed in claim 1, wherein the amount of coconut in the composition is between about 360-500 ml.

4. The composition as claimed in claim 1, wherein the composition is capable of producing a protein similar to collagen.

5. The composition as claimed in claim 4, wherein the composition is capable of removing scars from the skin by producing a protein similar to collagen.

6. The composition as claimed in claim 1, wherein the composition is prepared in a cauldron.

7. The composition as claimed in claim 6, wherein the cauldron is made up Bronze type of bell metal.

8. The composition as claimed in claim 6, wherein the cauldron comprising: a broad oval shaped open mouth,
a broad spherical bottom connecting to the broad oval shaped open mouth of the cauldron in which the composition is prepared and
a lid to cover the mouth of the cauldron.

9. The composition as claimed in claim 1, wherein the Shanku Padikaram is a form of read lead and has a chemical formula Triplumbic Tetroxide.

10. The method for treatment of burn with scar removal comprising the steps of: mixing Shanku Padikaram with coconut oil,
heating the mixture of Shanku Padikaram and coconut oil in the cauldron for 8-10 minutes,
cooling the mixture at room temperature and
application of mixture over affected area for a period of 2-3 months till the healing of the burn.
